(19) United States
(12) Patent Application Publication
(10) Pub. No.: US 2026/0000655 A1
(43) Pub. Date: Jan. 1, 2026

(54) PHARMACEUTICAL COMPOSITION FOR USE IN PREVENTION OR TREATMENT OF LIFESTYLE-RELATED DISEASE

(71) Applicants: NATIONAL UNIVERSITY CORPORATION KUMAMOTO UNIVERSITY, Kumamoto (JP); SCIENCE FARM LTD., Kumamoto (JP)

(72) Inventors: Eiichi ARAKI, Kumamoto (JP); Hiroshi TATEISHI, Kumamoto (JP); Tsugumasa TOMA, Kumamoto (JP); Mikako FUJITA, Kumamoto (JP); Yuiichi ARAKAKI, Kumamoto (JP); Tastuya KONDO, Kumamoto (JP); Nobukazu MIYAKAWA, Kumamoto (JP); Masami OTSUKA, Kumamoto (JP)

(73) Assignees: NATIONAL UNIVERSITY CORPORATION KUMAMOTO UNIVERSITY, Kumamoto (JP); SCIENCE FARM LTD., Kumamoto (JP)

(21) Appl. No.: 18/847,086

(22) PCT Filed: Mar. 15, 2023
(86) PCT No.: PCT/JP2023/010060

§ 371 Date: Jul. 17, 2025

(30) Foreign Application Priority Data
Mar. 16, 2022 (JP) 2022-041748

(51) Int. Cl. A61K 31/44; A61P 1/16; A61P 3/04; A61P 3/10

(52) U.S. Cl. CPC A61K 31/44; A61P 1/16; A61P 3/04; A61P 3/10

(57) ABSTRACT

A pharmaceutical composition for treating lifestyle-related diseases including type 2 diabetes and obesity-related disease. The pharmaceutical composition includes a compound represented by the following formula (1) or a salt thereof as an active ingredient.

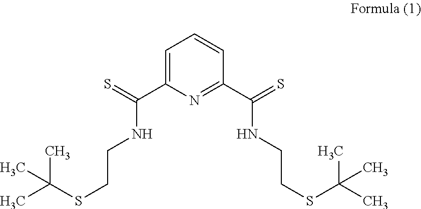 [SMILES: CC(C)(C)SCCNC(=S)c1cccc(C(=S)NCCSC(C)(C)C)n1]

### TECHNICAL FIELD

The present invention relates to a pharmaceutical composition used in a prevention or treatment of lifestyle-related disease.

### BACKGROUND ART

According to the International Diabetes Federation (IDF), the number of patient with diabetes worldwide has reached 463 million in 2019 and is still on the rise. In Japan, the number of patients has reached a record high of over 3.28 million, with most of them having type 2 diabetes. The 2018 Consensus Report by the American Diabetes Association (ADA) and the European Association for the Study of Diabetes (EASD) recommend, as the first step, a comprehensive lifestyle improvement (a dietary therapy, a exercise therapy, etc.) along with a treatment with metformin, which is a biguanide drug. For this reason, although a number of therapeutic drugs with new mechanisms of action have been launched on the market since 2010, metformin, which was developed in the 1960s, has been recommended as the drug of first choice in Europe and the United States. Also in Japan, metformin is the most commonly used diabetes treatment drug, accounting for 37% of prescriptions, although metformin is not recommended as the first-choice drug

It has been reported that a biguanide diabetes drug, which is an activator of AMPK (AMP-activated protein kinase), not only regulates blood glucose but also improves insulin resistance, reduces fat production, prevents a complication such as myocardial infarction, and the like. On the other hand, there remains a problem of the large dosage required for oral administration and the side effects resulting from such a large dosage. Thus, there has been a need to develop a drug that treats diabetes at a lower dosage than metformin.

The present inventors have disclosed that a pyridine derivative, HPH-15, is effective against fibrosis, systemic sclerosis, and inflammatory bowel disease (see Patent Literature 1 and Non-Patent Literature 1). Further, the present inventors have disclosed that HPH-15 has anti-herpes activity (see Non-Patent Literature 2), and that HPH-15 reduces the phosphorylation of Smad 3 induced by TGF-β, thereby preventing symptoms of skin fibrosis and collagen deposition (see Non-Patent Literature 3). However, the relationship between HPH-15 and the reduction of glucose and fat accumulation is not known.

### CITATION LIST

### Patent Literature

Patent Literature 1: WO2020/158890

### Non-Patent Literature

Non-Patent Literature 1: Vu Huy Luong et al., Arthritis Research & Therapy volume 20, Article number: 46 (2018)
Non-Patent Literature 2: Hosono T et al., Bioorg. Med. Chem. Lett., 2008, 18, 371-4.
Non-Patent Literature 3: Vu Huy Luong et al., Arthritis Research & Therapy, 2017, Vol. 86, issue 2, pp. 1-13

### SUMMARY OF THE INVENTION

### Technical Problem

An object of the present invention is to provide a pharmaceutical composition used in a prevention or treatment of lifestyle-related disease including type 2 diabetes and obesity-related disease.

### Means for Solving the Problem

As a result of intensive studies aimed at solving the above-mentioned problems, the present inventors have discovered that HPH-15 not only lowers a blood glucose level but also reduces fat accumulation, thereby completing the present invention.

Specifically, the present invention is as follows.
(1) A pharmaceutical composition used in a prevention or treatment of any one of lifestyle-related diseases selected from the group consisting of type 2 diabetes, a complication of type 2 diabetes, and fat-related disease, the pharmaceutical composition including a compound represented by the following formula (1) or a salt thereof as an active ingredient.

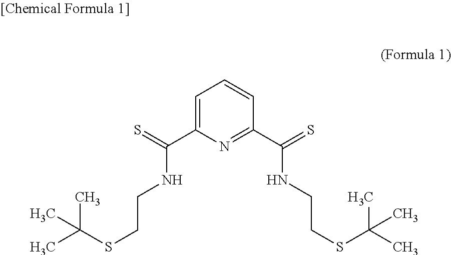 [SMILES: CC(C)(C)SCCNC(=S)c1cccc(C(=S)NCCSC(C)(C)C)n1]

(2) The pharmaceutical composition according to the above-mentioned (1), in which the lifestyle-related disease is the type 2 diabetes or the complication of type 2 diabetes, and the complication of type 2 diabetes is diabetic retinopathy, diabetic macular edema, neovascular glaucoma, diabetic nephropathy, diabetic foot ulcer, or diabetic neuropathy caused by the type 2 diabetes.
(3) The pharmaceutical composition according to the above-mentioned (1), in which the lifestyle-related disease is the type 2 diabetes.
(4) The pharmaceutical composition according to the above-mentioned (1), in which the lifestyle-related disease is the fat-related disease, and the fat-related disease is fatty liver disease, subcutaneous fat obesity, visceral fat obesity, hyperlipidemia, atherosclerosis, coronary artery disease, macroangiopathy, cerebral infarction, or ischemic heart disease.
(5) The pharmaceutical composition according to the above-mentioned (4), in which the fatty liver disease is nonalcoholic steatohepatitis (NASH) or nonalcoholic fatty liver (NAFL).

Further, other aspects of the present invention are as follows.
(a) A use of a compound represented by the above-mentioned formula (1) or a salt thereof in the production of a pharmaceutical composition for preventing or treating any one of lifestyle-related diseases selected from the group consisting of type 2 diabetes, a complication of type 2 diabetes, and fat-related disease.
(b) A compound represented by the above-mentioned formula (1) or a salt thereof used in a prevention or treatment of any one of lifestyle-related diseases selected from the group consisting of type 2 diabetes, a diabetic complication, and fat-related disease.
(c) A method for treating any one of lifestyle-related diseases selected from the group consisting of type 2 diabetes, a complication of type 2 diabetes, and fat-related disease, the method including administering a compound represented by the above-mentioned formula (1) or a salt thereof to a subject.

### Advantageous Effect

The pharmaceutical composition of the present invention makes it possible to prevent or treat the lifestyle-related disease including the type 2 diabetes, the complication of type 2 diabetes, and the fat-related disease such as nonalcoholic fatty liver disease.

### BRIEF DESCRIPTION OF DRAWINGS

FIG. 1 is diagram showing results of examining activated AMPK relative to AMPK (p-AMPK/AMPK) after HPH-15, metformin, or insulin is administered to a myotube cell model L6-GLUT4myc, a liver cell model HepG2, and an adipocyte model 3T3-L1 in Example 1.

FIG. 2 is diagram showing results of examining the amount of GLUT4 localized in the membrane after HPH-15, metformin, or insulin is administered to the myotube cell model L6-GLUT4myc in Example 2.

FIG. 3 is diagram showing results of examining the amount of glucose uptake into cells after HPH-15, metformin, or insulin is administered to the myotube cell model L6-GLUT4myc, the liver cell model HepG2 cells, and the adipocyte model 3T3-L1 in Example 3.

FIG. 4 is diagram showing results of examining the amount of lactic acid produced after HPH-15 or metformin is administered to the myotube cell model L6-GLUT4myc, the liver cell model HepG2 cells, and the adipocyte model 3T3-L1 in Example 4.

FIG. 5A is diagram showing results of microscopic observation of the fixed liver cell model HepG2 cells stained with Oil Red O after administration of HPH-15 or metformin in the presence of glucose in Example 5.

FIG. 5B is a diagram showing results of examining the amount of neutral fat after HPH-15 or metformin is administered in the presence of glucose to the liver cell model HepG2 cells in Example 5.

FIG. 6 is a diagram showing results of examining the cytotoxicity, using an MTT reagent, of the myotube cell model L6-GLUT4myc, the liver cell model HepG2 cells, and the adipocyte model 3T3-L1 after administration of HPH-15 or metformin in Example 6.

FIG. 7A is a diagram showing results of examining random blood glucose levels after HPH-15 or metformin is administered to high-fat diet-induced obesity model mice in Example 7.

FIG. 7B is a diagram showing results of examining fasting blood glucose levels after HPH-15 or metformin is administered to the high-fat diet-induced obesity model mice in Example 7.

FIG. 7C is a diagram showing results of examining the body weight after HPH-15 or metformin is administered to the high-fat diet-induced obesity model mice in Example 7.

FIG. 8 is a diagram showing results of examining blood glucose levels after HPH-15 or metformin is added and then glucose is administered to the high-fat diet-induced obesity model mice in Example 8.

FIG. 9 is a diagram showing results of examining glucose metabolism after HPH-15 or metformin is added and then pyruvic acid is administered to the high-fat diet-induced obesity model mice in Example 9.

FIG. 10 is a diagram showing results of examining the weights of the liver, muscle, subcutaneous fat, and epididymis after HPH-15 or metformin is administered to the high-fat diet-induced obesity model mice in Example 10.

FIG. 11 is a diagram showing results of performing a blood test after HPH-15 or metformin is administered to the high-fat diet-induced obesity model mice in Example 11.

FIG. 12A is a diagram showing results of microscopic observation of the liver tissue stained with hematoxylin and eosin (H&E staining) after HPH-15 or metformin is administered to the high-fat diet-induced obesity model mice in Example 12.

FIG. 12B is a diagram showing results of microscopic observation of the epididymal adipose tissue stained with H&E after HPH-15 or metformin is administered to the high-fat diet-induced obesity model mice in Example 12.

FIG. 12C is a diagram showing results of microscopic observation of the muscle tissue stained with H&E after HPH-15 or metformin is administered to the high-fat diet-induced obesity model mice in Example 12.

FIG. 13 is a diagram showing results of examining AMPK activity in the liver, subcutaneous fat, and muscle after HPH-15 or metformin is administered to the high-fat diet-induced obesity model mice in Example 13.

FIG. 14A is a diagram showing results of microscopic observation of the liver tissue and epididymal adipose tissue stained with Sirius Red after HPH-15 or metformin is administered to the high-fat diet-induced obesity model mice in Example 14.

FIG. 14B is a diagram showing results of performing Western blotting using protein solutions which are prepared after homogenizing tissue pieces collected from the liver tissue and the epididymal adipose tissue after HPH-15 or metformin is administered to the high-fat diet-induced obesity model mice in Example 14.

FIG. 14C is a diagram showing results of calculating the amount of collagen using ImageJ after performing Western blotting with the liver tissue in Example 14.

FIG. 14D is a diagram showing results of calculating the amount of collagen using ImageJ after performing Western blotting with the epididymal adipose tissue in Example 14.

### MODES FOR CARRYING OUT THE INVENTION

A pharmaceutical composition of the present invention includes a compound represented by the following formula (1) or a salt thereof as an active ingredient and is used for preventing or treating lifestyle-related disease. Hereinafter the pharmaceutical composition of the present invention is also referred to as “present pharmaceutical composition”. Note that the compound represented by the formula (1) is known as histidine-pyridine-histidine (HPH)-15, and is also referred to simply as “HPH-15” below.

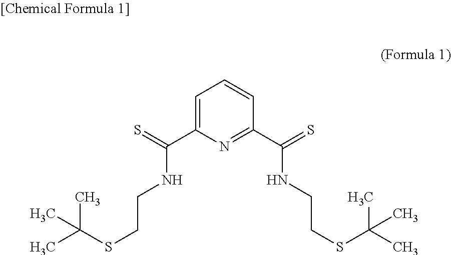 [SMILES: CC(C)(C)SCCNC(=S)c1cccc(C(=S)NCCSC(C)(C)C)n1]

HPH-15 can be synthesized by the method described in Non-Patent Literature 1. Briefly, HPH-15 can be synthesized by converting 2,6-pyridine dicarboxylic acid to the carboxylic acid chloride by thionyl chloride, reacting the carboxylic acid chloride with 2-tert-butyl sulfenylethylamine hydrochloride, and subjecting the resulting substance to thioamidation using Lawesson's reagent.

The “salt” in the term “compound represented by the above-mentioned formula (1) or a salt thereof” is not particularly limited as long as it is pharmacologically acceptable. However, examples of the salt include, but are not limited to, a salt with an inorganic base such as sodium, potassium, magnesium, calcium, or aluminum, and a salt with an organic base such as methylamine, ethylamine, or ethanolamine. Further, the above-mentioned salt may be an acid addition salt, and examples thereof include an acid addition salt with a mineral acid such as hydrochloric acid, hydrobromic acid, hydroiodic acid, sulfuric acid, nitric acid, or phosphoric acid, and an acid addition salt with an organic acid such as formic acid, acetic acid, trifluoroacetic acid, propionic acid, oxalic acid, malonic acid, succinic acid, fumaric acid, maleic acid, lactic acid, malic acid, tartaric acid, citric acid, methanesulfonic acid, or ethanesulfonic acid.

In the present specification, examples of the lifestyle-related disease include type 2 diabetes (non-insulin-dependent diabetes mellitus), a complication due to type 2 diabetes such as diabetic retinopathy, diabetic macular edema, neovascular glaucoma, diabetic nephropathy, diabetic foot ulcer, or diabetic neuropathy, and fat-related disease such as fatty liver disease, subcutaneous fat obesity, visceral fat obesity, hyperlipidemia, atherosclerosis, abdominal aortic aneurysm, coronary artery disease, macroangiopathy, cerebral infarction, or ischemic heart disease. Further, examples of the above-mentioned fatty liver disease include nonalcoholic fatty liver disease (NAFLD) such as nonalcoholic steatohepatitis (NASH) or nonalcoholic fatty liver (NAFL), and metabolic dysfunction-associated fatty liver disease (MAFLD). Further, as a preferable example of the above-mentioned fat-related disease such as fatty liver disease, the fat-related disease such as fatty liver disease caused by the above-mentioned type 2 diabetes can be mentioned. As a preferable example of the above-mentioned type 2 diabetes, the type 2 diabetes caused by the above-mentioned fat-related disease can be mentioned. The above-mentioned fat-related disease also includes the above-mentioned fatty liver disease, preferably liver cirrhosis or hepatocellular carcinoma caused by NASH.

The subcutaneous fat in the subcutaneous fat obesity is not particularly limited as long as it is fat under the skin, and examples thereof include fat present under the skin of the abdomen, lower back, buttocks, and thighs. Further, the visceral fat in the visceral fat obesity is not particularly limited as long as it is fat of the visceral organs, and examples thereof include fat of the liver, intestines, kidneys, and the like.

In the present specification, the term “prevention” means preventing or delaying the onset of disease by administering the present pharmaceutical composition to a subject before the onset of the disease, and does not necessarily mean completely blocking the onset of the disease. Specifically, this means that, when the present pharmaceutical composition is administered to a subject, the glucose concentration in the blood is favorably adjusted to a normal level or a state close to a normal level, fat accumulation is reduced, thereby slowing or delaying the progression to type 2 diabetes, the complication of type 2 diabetes, or the fat-related disease, or the like. Note that the onset of disease means that a symptom of the disease appears in the body.

In the present specification, the term “treatment” means alleviating a symptom of the disease by administering the present pharmaceutical composition to a subject after the onset of the disease, and does not necessarily mean completely curing the symptom of the disease. Specifically, if administration of the present pharmaceutical composition can improve a clinical state of the disease or one or more of biological symptoms of the clinical state, it can be said that the pharmaceutical composition can be used to treat type 2 diabetes, the complications of type 2 diabetes, or the fat-related disease.

The present pharmaceutical composition can be administered to a mammal. Example of the mammal include human, a dog, a cat, a monkey, a cow, a horse, a mouse, a rat, a hamster, a guinea pig, a rabbit, a goat, a pig, and sheep.

Further, the present pharmaceutical composition can also be used as an agent for promoting glucose uptake into cells. HPH-15 promotes membrane translocation of the glucose transporter GLUT4 (glucose transporter type 4) and thus has an effect of increasing glucose uptake into cells. This allows GLUT4 to appropriately promote glucose uptake into cells in response to insulin, making it possible to maintain the blood glucose level at an appropriate level. Examples of the cells that take up glucose include cells of the muscle, the liver, and the subcutaneous fat.

Further, the present pharmaceutical composition can be used as an agent for reducing fat, particularly as an agent for reducing fat in the liver and subcutaneous fat. HPH-15 has an effect of reducing the percentage of fat as well as the number of fat cells in the liver and subcutaneous fat. Thus, the present pharmaceutical composition makes it possible to reduce fat.

The present pharmaceutical composition may further include, as necessary, a pharmaceutically acceptable conventional additive such as a carrier, a binder, a stabilizer, an excipient, a diluent, a pH buffer, a disintegrant, a tonicity agent, a coating agent, a solubilizer, a lubricant, a solubilizing agent, a lubricant, a flavoring agent, a sweetener, a solvent, a gelling agent, or a nutrient. Specific examples of such an additive include water, physiological saline, animal fat and oil, vegetable oil, lactose, starch, gelatin, crystalline cellulose, gum, talc, magnesium stearate, hydroxypropyl cellulose, polyalkylene glycol, polyvinyl alcohol, and glycerin.

The present pharmaceutical composition can be administered orally in the form of a powder, a granule, a tablet, a capsule, a syrup, a suspension, or the like, or parenterally injected in the form of a solution, an emulsion, a suspension, or the like (e.g., subcutaneous injection, intravenous injection, and intramuscular injection) or parenterally administered through intranasal route in the form of a spray. The daily dose can be adjusted depending on the method of administration, the symptom of the subject, the body weight of the subject, the age of the subject, and the like. For example, as an active ingredient, about 0.01 mg to 500 mg, preferably about 0.1 mg to 300 mg, more preferably about 0.05 mg to 200 mg, per 1 kg body weight of human or a non-human mammal can be administered once or in two or more divided doses.

### EXAMPLES

The present invention is described in more detail below with reference to examples. However, the technical scope of the present invention is not limited to these examples.

### [Example 1] Activation of AMPK

The tissues involved in glucose uptake and glucose metabolism are mainly the muscle, the liver, and the fat. Thus, using a cell model of each tissue, the relationship between HPH-15 and activation of adenosine monophosphate-activated protein kinase (AMPK) was examined. Note that AMPK is known as a therapeutic target for diabetes. AMPK acts as an energy sensor, and when it detects a decrease in ATP serving as an intracellular energy source, it suppresses the synthesis of glucose, fats, and proteins and produces ATP. Exercise therapy for diabetes is based on this mechanism. Further, AMPK has been drawing attention as a factor that improves insulin resistance.

As a muscle cell model to be used, rat myoblast cell line L6-GLUT4myc (myoblasts expressing myc-tagged GLUT4) was cultured in a minimal essential medium a-MEM (10% FBS) and differentiated into myotubes. Differentiation into myotubes was performed by culturing L6-GLUT4 cells in a-MEM (10% FBS) and then changing the medium to a-MEM (2% FBS), followed by culturing for 6 days. Human HepG2 cell line was used as a liver cell model. As an adipocyte model to be used, mouse 3T3-L1 preadipocyte cell line was differentiated into adipocytes. Differentiation into adipocytes was performed as follows. First, 3T3-L1 cell line was cultured in DMEM (10% FBS) for 2 days, and then 3-isobutyl-1-methylxanthine (0.5 mM/L), dexamethasone (1 mM), and insulin (1 mg/L) were added to the culture, followed by culturing for 2 days. The medium was then replaced with DMEM (10% FBS) supplemented with insulin (1 mg/L), and the cells were further cultured for 3 days.

The amount of activated AMPK (phosphorylated AMPK: phospho-AMPK (p-AMPK)) was examined by Western blotting. The cells used were the above-mentioned myotube cell model L6-GLUT4myc, liver cell model HepG2 cells, and adipocyte model 3T3-L1. Each type of cell was seeded onto a 24-well plate (1×10⁵ cells/well, 500 mL of culture liquid) and cultured overnight. As a culture medium, the medium for the differentiation process described above was used for the myotube cell model L6-GLUT4myc and the adipocyte model 3T3-L1, while DMEM (10% FBS) was used for the liver cell model HepG2 cells. Then, a DMSO solution (1%) containing HPH-15 (final concentration: 10 μM or 50 μM) synthesized based on the method described in the above-mentioned Non-Patent Literature 1, metformin (final concentration: 2 mM, FUJIFILM Wako Pure Chemical Corp.), or insulin (final concentration: 100 nM, FUJIFILM Wako Pure Chemical Corp.), or a DMSO solution (1%) alone as a control was added to the medium, and the cells were further cultured for 24 hours. The cells were disrupted in RIPA (Radio-Immunoprecipitation Assay) buffer, and the disruption solution was boiled at 100° C. for 10 minutes to lyse the cells. The resulting cell lysates were subjected to electrophoresis, transferred to a membrane (MilliporeSigma), and reacted with anti-phospho-AMPK (Thr172) antibodies (Cell Signaling Technology, Inc.) as the primary antibodies. Detection was performed using ImmunoStar LD (FUJIFILM Wako Pure Chemical Corp.). Note that metformin was developed in the 1960s as a diabetes therapeutic drug that activates AMPK and metabolizes excess glucose, and metformin is still used today as the first-choice drug for treating diabetes. Thus, metformin was used for a comparison as a drug having the same mechanism of action. Further, insulin was used as a control having a signal different from AMPK.

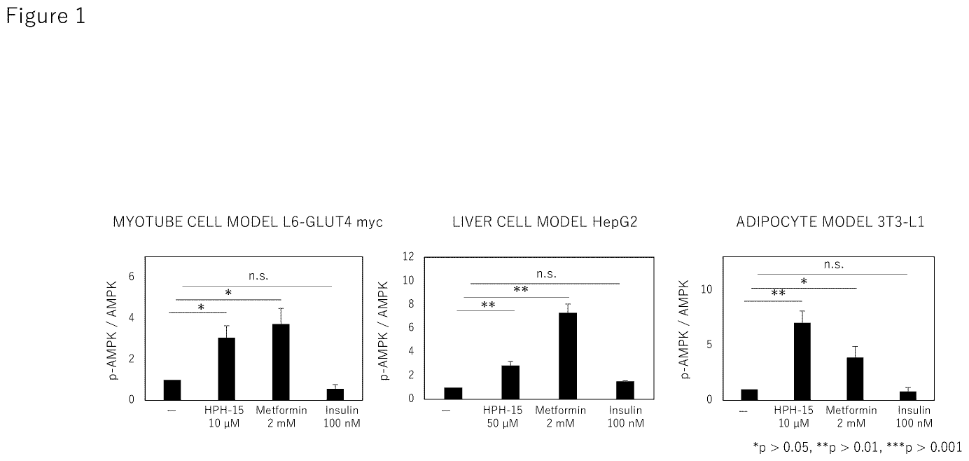

Next, bands of AMPK and p-AMPK were quantified using ImageJ to determine a ratio of activated AMPK relative to AMPK (p-AMPK/AMPK). FIG. 1 shows resulting graphs of the relative values of p-AMPK/AMPK when HPH-15, metformin, or insulin was added, with the intracellular p-AMPK/AMPK when only control DMSO was added being taken as 1.

As shown in FIG. 1, it was found that, in all cell lines, including the myotube cell model L6-GLUT4myc, the liver cell model HepG2 cells, and the adipocyte model 3T3-L1, HPH-15 activated AMPK in the same manner as metformin, and the effect was particularly remarkable in the adipocytes. Further, it was found that the use of HPH-15 activated AMPK at low concentrations. Specifically, in the case of the myotube cell model L6-GLUT4myc, HPH-15 activated AMPK by approximately 75% compared to when metformin was used even though the concentration of HPH-15 was as low as 1/200th that of metformin. In the case of the liver cell model HepG2, HPH-15 activated AMPK by approximately 40% compared to when metformin was used even though the concentration of HPH-15 was as low as 1/40th that of metformin. In the case of the adipocyte model 3T3-L1, HPH-15 activated AMPK by approximately 170% compared to when metformin was used even though the concentration of HPH-15 was as low as 1/200th that of metformin.

### [Example 2] Promotion of GLUT4 Membrane Translocation

GLUT4 (glucose transporter type 4) is involved in the uptake of glucose into cells. Thus, the relationship between HPH-15 and the membrane translocation of GLUT4 was examined using the myotube cell model L6-GLUT4myc.

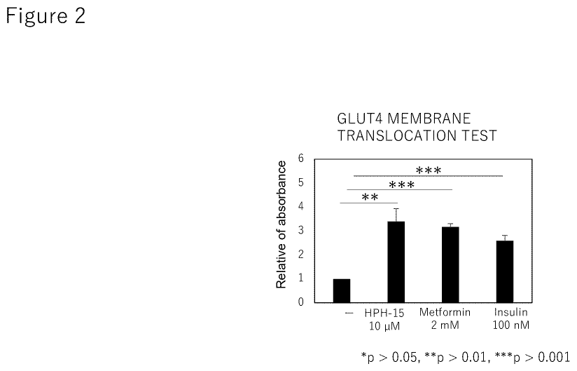

The myotube cell model L6-GLUT4myc cells used in Example 1 were seeded onto a 24-well plate (1×10⁵ cells/well, 500 mL of culture liquid) and cultured overnight. Then, a DMSO solution (1%) containing HPH-15 (final concentration: 10 μM), metformin (final concentration: 2 mM), or insulin (final concentration: 100 nM) was added to the medium, and the cells were further cultured for 24 hours. As a control, only DMSO solution (1%) was added, and the cells were cultured for 24 hours in the same manner. Next, each cell was fixed for 20 minutes with a 3% paraformaldehyde solution (Tokyo Chemical Industry Co., Ltd.). The reaction was stopped with an 1% glycine solution (Merck KGaA), and blocking was performed for 15 minutes using a PBS solution containing 5% goat serum (FUJIFILM Wako Pure Chemical Corp.). To this, a PBS solution containing anti-Myc antibodies (Medical & Biological Laboratories Co., Ltd.) containing 1% BSA was added as the primary antibodies, and the reaction was performed for 1 hour. Then, the cells were reacted with HRP-conjugated goat anti-mouse IgG antibodies as secondary antibodies for 30 minutes. Detection was performed using OPD reagent (o-phenylenediamine dihydrochloride: FUJIFILM Wako Pure Chemical Corp.), and the amount of GLUT4 was evaluated by measuring absorbance (492 nm). FIG. 2 shows a resulting graph of the relative values of the amount of GLUT4 localized in the membrane when HPH-15, metformin, or insulin was administered, with the amount of GLUT4 localized in the cell membrane when only DMSO was administered being taken as 1.

As shown in FIG. 2, it was found that HPH-15 promoted membrane translocation of GLUT4 to the same extent as metformin or insulin at a concentration as low as 1/200th that of metformin.

### [Example 3] Glucose Uptake

The uptake of glucose into cells by HPH-15 was examined using the myotube cell model L6-GLUT4myc, the liver cell model HepG2 cells, and the adipocyte model 3T3-L1.

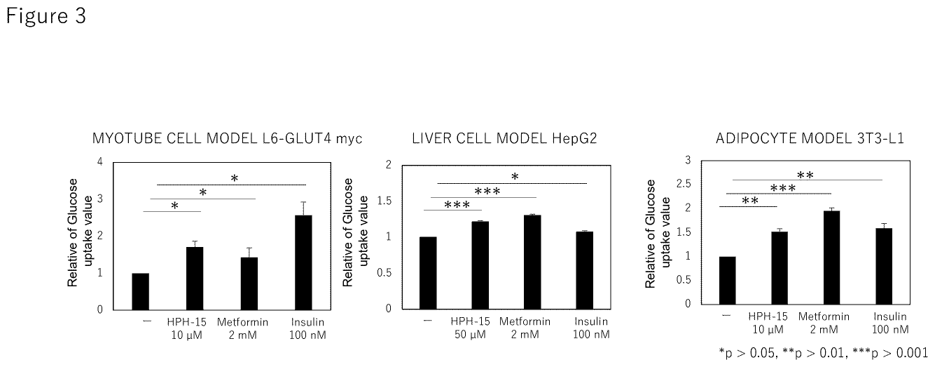

The myotube cell model L6-GLUT4myc, the liver cell model HepG2 cells, and the adipocyte model 3T3-L1 used in Example 1 were each seeded onto a 24-well plate (1×10⁵ cells/well, 500 mL of culture liquid) and cultured overnight. Then, a DMSO solution (1%) containing HPH-15 (final concentration: 10 μM or 50 μM), metformin (final concentration: 2 mM), or insulin (final concentration: 100 nM) was added to the medium, and the cells were further cultured for 24 hours. As a control, only DMSO solution (1%) was added, and the cells were cultured for 24 hours in the same manner. Next, the cells were washed with KRPH buffer (containing 2% BSA) and cultured in the presence of 2-deoxyglucose (final concentration: 2 mM, FUJIFILM Wako Pure Chemical Corp.) for 20 minutes at 37° C. The uptake of 2-deoxyglucose was stopped by washing the cells with cold PBS, and the amount of glucose taken up into the cells was measured using a Glucose Cellular Uptake Measurement Kit (Cosmo Bio). FIG. 3 shows resulting graphs of the relative values of the glucose amount when HPH-15, metformin, or insulin was administered, with the amount of glucose taken up into the cells when only control DMSO was administered being taken as 1.

As shown in FIG. 3, it was found that HPH-15 increased the glucose uptake amount to the same extent as or to a greater extent than metformin.

### [Example 4] Production of lactic acid

There is a concern that a therapeutic drug of diabetes may cause lactic acidosis due to the production of lactic acid, requiring caution, especially for patients with chronic renal failure or the elderly. Therefore, the production of lactic acid when HPH-15 was used was examined.

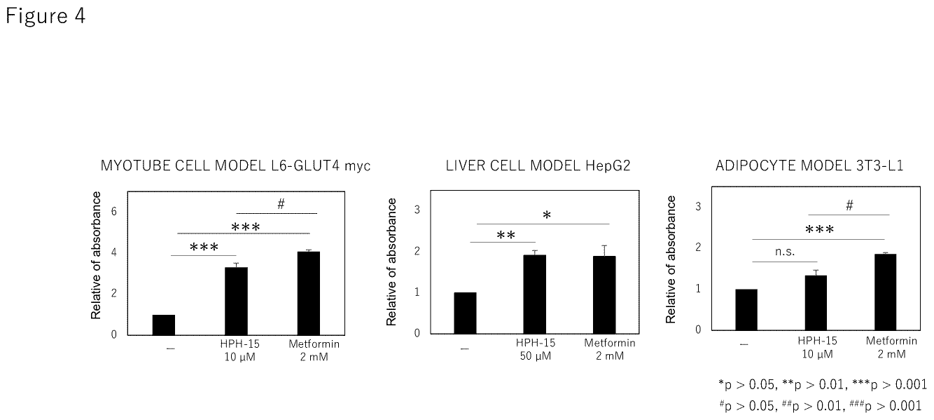

The myotube cell model L6-GLUT4myc, the liver cell model HepG2 cells, and the adipocyte model 3T3-L1 used in Example 1 were each seeded onto a 24-well plate (1×10⁵ cells/well, 500 mL of culture liquid) and cultured overnight. Then, a DMSO solution (1%) containing HPH-15 (final concentration: 10 μM or 50 μM) or metformin (final concentration: 2 mM) was added to the medium, and the cells were further cultured for 24 hours. As a control, only DMSO solution (1%) was added, and the cells were cultured for 24 hours in the same manner. Next, the amount of lactic acid accumulated in the cells was measured using the Lactate Assay Kit-WST (Dojindo Laboratories). FIG. 4 shows resulting graphs of the relative values of the amount of lactic acid in each cell when HPH-15 or metformin was administered, with the amount of lactic acid measured in the cells when only control DMSO was administered being taken as 1.

As shown in FIG. 4, it was found that the production of lactic acid by HPH-15 was equal to or less than that by metformin. The diabetes therapeutic drugs sold to date include a biguanide drug such as buformin or phenformin. However, due to a series of cases of lactic acidosis as a side effect, these two drugs were discontinued. As a result, metformin, which produces less lactic acid, is used as the drug of first choice. Since HPH-15 produces lactic acid at a level same as or lower than metformin, HPH-15 is thought to have low lactic acidosis as a side effect. Further, HPH-15 can be used in patients with reduced renal function by controlling the dosage.

### [Example 5] Effect on Neutral Fat Accumulation

Many patients with type 2 diabetes have fat accumulation in the visceral organs. Thus, it was examined whether HPH-15 had an inhibitory effect on neutral fat accumulation in the liver.

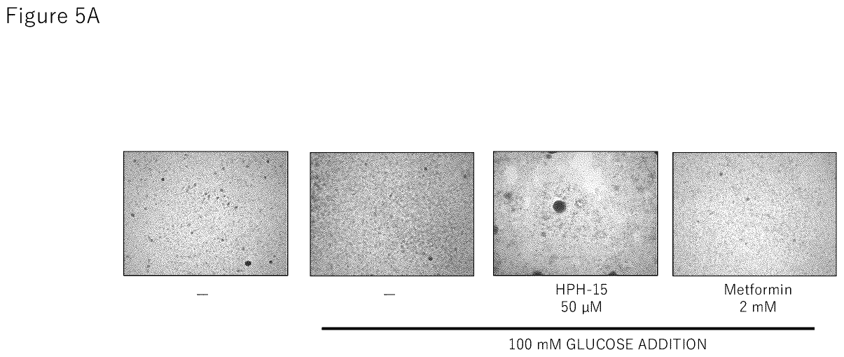

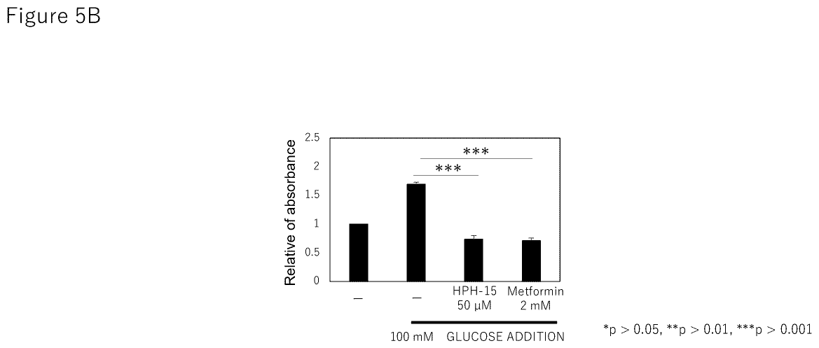

The liver cell model HepG2 cells used in Example 1 were seeded onto a 24-well plate (1×10⁵ cells/well, 500 mL of culture liquid) and cultured overnight. Then, a DMSO solution (1%) containing HPH-15 (final concentration: 50 μM) or metformin (final concentration: 2 mM) was added to the medium, and the cells were further cultured for 24 hours. As a control, only DMSO solution (1%) was added, and the cells were cultured for 24 hours in the same manner. Next, glucose (100 mM) was added to the medium, and the cells were further cultured for 24 hours. Then, the cells were fixed using 4% paraformaldehyde for 10 minutes. The fixed cells were added with an Oil Red O staining solution (Nacalai Tesque, Inc.) and shaken for 15 minutes. After washing with a 60% aqueous isopropanol solution, cell images were observed using a microscope. The results are shown in FIG. 5A. Further, the amount of neutral fat in the cells was determined by dissolving the stained cells in 100% isopropanol and measuring the absorbance (492 nm). FIG. 5B shows a resulting graph of the relative values of the amount of neutral fat when DMSO alone, HPH-15, or metformin was administered in the presence of glucose, with the amount of neutral fat in the cells when only control DMSO was administered (in the absence of glucose) being taken as 1.

As shown in FIG. 5A and FIG. 5B, it was found that, in the liver cell model, HPH-15 had an inhibitory effect on neutral fat accumulation in the presence of high glucose in the same manner as metformin. Thus, HPH-15 is thought to have preventive or therapeutic effects on the fat-related disease, especially the fatty liver disease.

### [Example 6] Cytotoxicity

It was examined whether using HPH-15 as a drug caused any cytotoxicity.

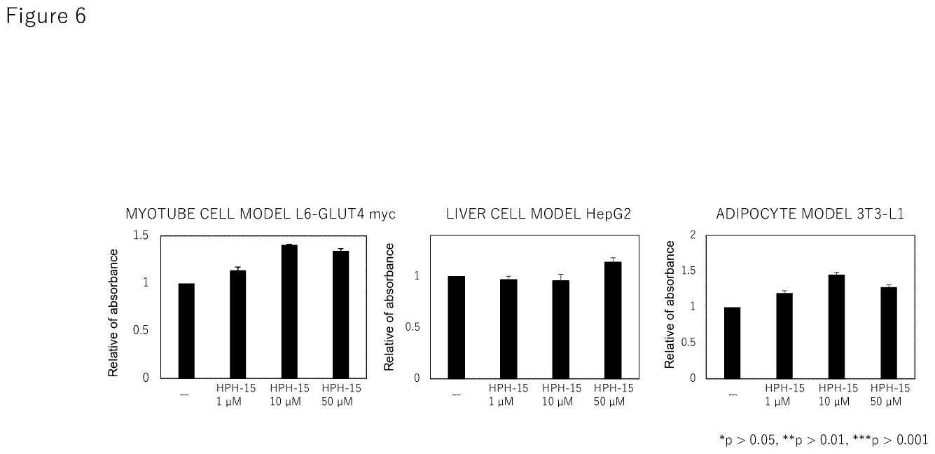

The myotube cell model L6-GLUT4myc, the liver cell model HepG2 cells, and the adipocyte model 3T3-L1 used in Example 1 were each seeded onto a 24-well plate (1×10⁵ cells/well, 500 mL of culture liquid) and cultured overnight. Then, a DMSO solution (1%) containing HPH-15 (final concentration: 1 μM, 10 μM or 50 μM) was added to the medium, and the cells were further cultured for 24 hours. As a control, only DMSO solution (1%) was added, and the cells were cultured for 24 hours in the same manner. Next, 100 mL of MTT reagent (3-(4,5-dimethyl-2-thiazolyl)-2,5-diphenyl-2H-tetrazolium bromide: 1.1 mg/mL: Dojindo Laboratories) was added to each well, and the cells were further cultured for 4 hours. After collecting the precipitated crystals, the supernatant was removed and DMSO (100 mL) was added to completely dissolve the crystals. Cytotoxicity was evaluated by measuring the absorbance (550 nm) of the solution and determining the number of viable cells. FIG. 6 shows resulting graphs of the relative values of the number of viable cell when DMSO alone or various concentrations of HPH-15 were administered, with the number of viable cells when only control DMSO was administered being taken as 1.

As shown in FIG. 6, no cytotoxicity was confirmed in the myotube cell model L6-GLUT4myc, the liver cell model HepG2 cells, or the adipocyte model 3T3-L1 by adding HPH-15 at various concentrations.

### [Example 7] Blood Glucose Lowering Effect in Mouse

In Example 7, mice were used instead of the cells, which were used up to Example 6, to examine the blood glucose lowering effect of HPH-15.

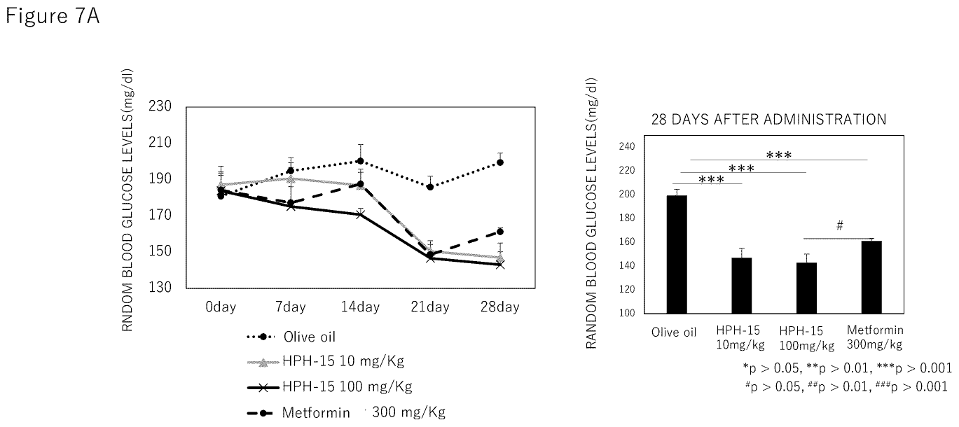

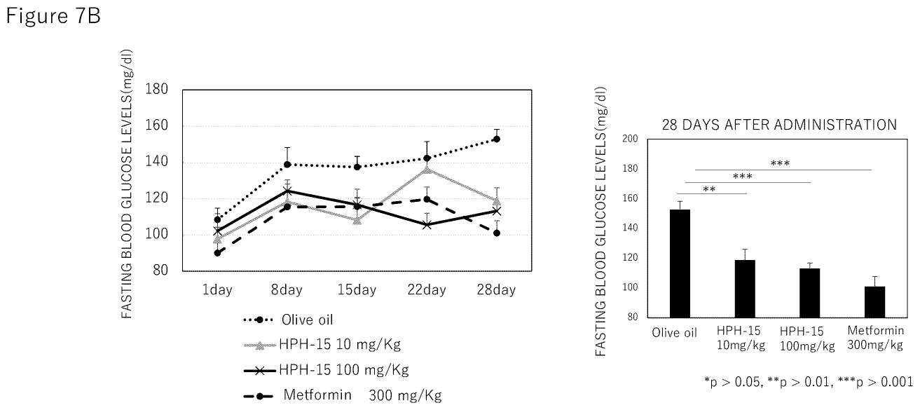

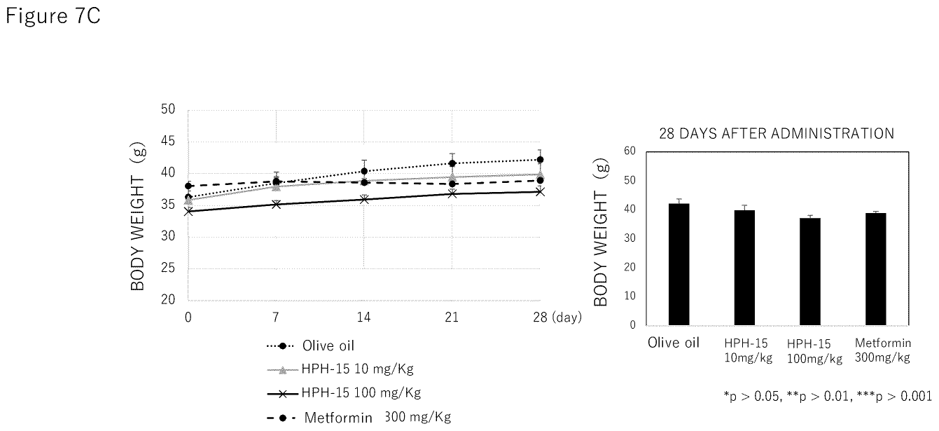

Four-week-old C57BL/J mice (CLEA Japan, Inc.) were fed a high-fat diet HFD (CLEA Japan, Inc.) for two months to prepare high-fat diet-induced obesity model mice. Then, HPH-15 (10 mg/kg or 100 mg/kg, 200 mL) dissolved in olive oil (Kenei Pharmaceutical Co., Ltd.) or metformin (300 mg/kg, 200 mL) was orally administered once a day for 28 days. Olive oil alone was administered as a control. Body weight, random blood glucose levels, and fasting blood glucose levels were measured every 7 days using a blood glucose meter Glutest Aqua (Sanwa Kagaku Kenkyusho Co., Ltd.). Fasting blood glucose levels were measured after 12-hour fasting. Note that, during the drug administration period, the mice were fed the high-fat diet HFD. The results of random blood glucose levels (mg/dl) are shown in FIG. 7A, and the results of fasting blood glucose levels (mg/dl) are shown in FIG. 7B.

As shown in FIG. 7A and FIG. 7B, it was found that HPH-15, not only when administered at 100 mg/kg, but even when administered at only 10 mg/kg, had the effect of lowering random blood glucose levels and fasting blood glucose levels in the same manner as metformin administered at 300 mg/kg. Note that no significant differences were observed in the changes in body weight (g) during the test period (FIG. 7C).

### [Example 8] Glucose Loading Test in Mouse

The effect of HPH-15 on the change in blood glucose levels in the presence of glucose was examined using mice.

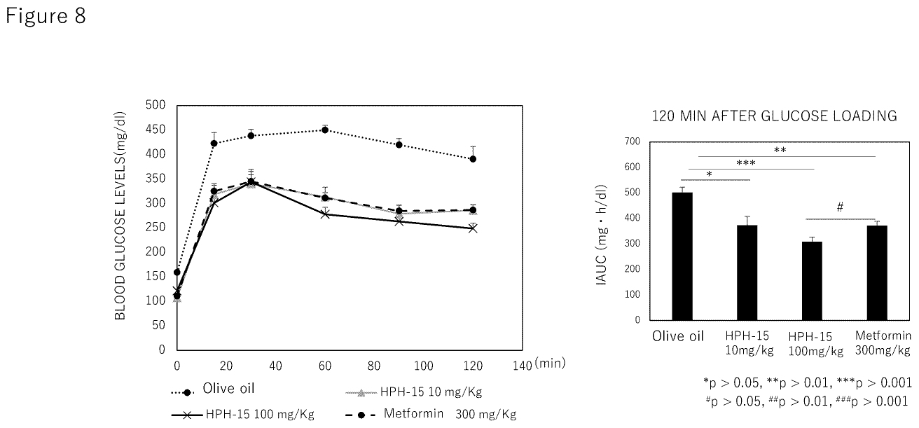

High-fat diet-induced obesity model mice were prepared in the same manner as in Example 7. Then, HPH-15 (10 mg/kg or 100 mg/kg, 200 mL) dissolved in olive oil or metformin (300 mg/kg, 200 mL) was orally administered once a day for 28 days. Olive oil alone was administered as a control. After 28 days of administration of each, glucose was intraperitoneally administered (0.5 g/kg) to the high-fat diet-induced obesity model mice, and blood glucose levels (mg/dl) were measured at 0, 15, 30, 60, 90, and 120 minutes after administration using a blood glucose meter Glutest Aqua (Sanwa Kagaku Kenkyusho Co., Ltd.). The measurement results of blood glucose levels at each time point are shown in FIG. 8. In FIG. 8, the left panel shows the blood glucose levels (mg/dl) at 0, 15, 30, 60, 90, and 120 minutes after glucose administration, and the right panel shows the incremental area under the curve of the blood glucose levels (IAUC: mg·h/dl) at 120 minutes after glucose administration (glucose loading).

As shown in FIG. 8, glucose metabolism was significantly improved when HPH-15 was administered compared to olive oil.

### [Example 9] Pyruvic Acid Loading Test in Mouse

The effect of HPH-15 on the changes in blood glucose levels in the presence of pyruvic acid involving in gluconeogenesis was examined using mice.

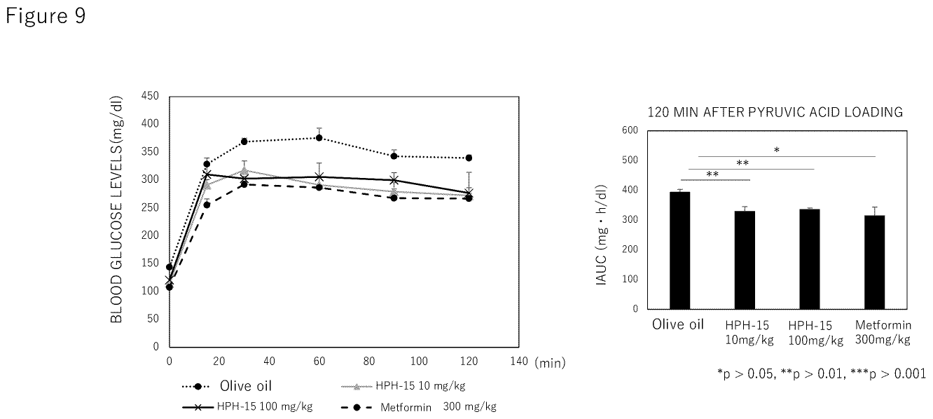

High-fat diet-induced obesity model mice were prepared in the same manner as in Example 7. Then, HPH-15 (10 mg/kg or 100 mg/kg, 200 mL) dissolved in olive oil or metformin (300 mg/kg, 200 mL) was orally administered once a day for 28 days. Olive oil alone was administered as a control. After 28 days of administration of each, pyruvic acid was intraperitoneally administered (2 g/kg) to the high-fat diet-induced obesity model mice, and blood glucose levels (mg/dl) were measured at 0, 15, 30, 60, 90, and 120 minutes after administration using a blood glucose meter Glutest Aqua (Sanwa Kagaku Kenkyusho Co., Ltd.). The measurement results of blood glucose levels at each time point are shown in FIG. 9. In FIG. 9, the left panel shows the blood glucose levels (mg/dl) at 0, 15, 30, 60, 90, and 120 minutes after pyruvic acid administration, and the right panel shows the incremental area under the curve of the blood glucose levels (IAUC: mg· h/dl) at 120 minutes after pyruvic acid administration (pyruvic acid loading).

As shown in FIG. 9, the blood glucose levels were significantly lowered when HPH-15 was administered compared to olive oil. This confirmed that administration of HPH-15 caused an effect of reducing gluconeogenesis by AMPK, thereby reducing an increase in the blood glucose even when gluconeogenesis is induced by the addition of pyruvic acid.

### [Example 10] Organ Weight in Mouse

The changes in organ weights when HPH-15 was administered were examined.

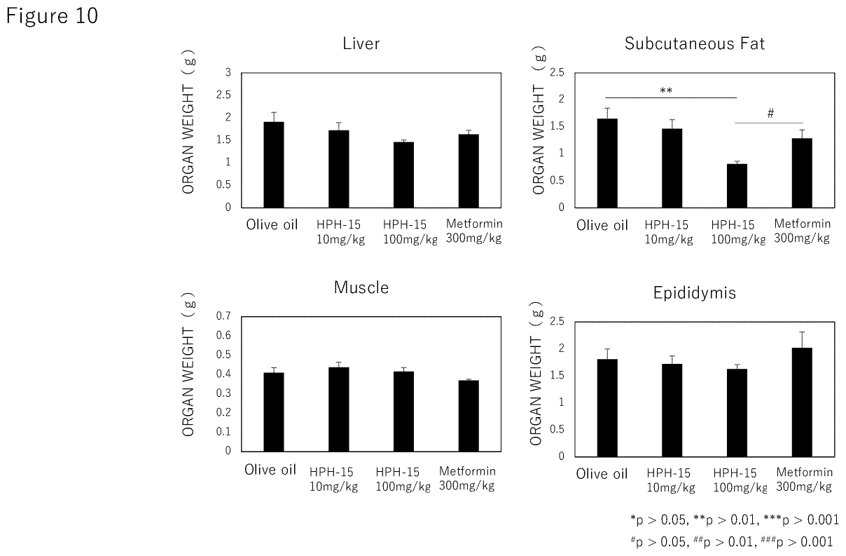

High-fat diet-induced obesity model mice were prepared in the same manner as in Example 7. Then, HPH-15 (10 mg/kg or 100 mg/kg, 200 mL) dissolved in olive oil or metformin (300 mg/kg, 200 mL) was orally administered once a day for 28 days. Olive oil alone was administered as a control. After 28 days of administration of each, the liver, muscle, subcutaneous fat, and epididymis of the high-fat diet-induced obesity model mice were excised and weighed. The weight (g) of each organ is shown in FIG. 10.

As shown in FIG. 10, in the liver, muscle, and epididymis, no significant differences were observed when HPH-15 or metformin was administered compared to olive oil. On the other hand, in the subcutaneous fat, when HPH-15 was administered at 100 mg/kg, the weight was reduced by approximately 50% compared to olive oil. These results demonstrated that HPH-15, unlike the metformin, had an effect of reducing the subcutaneous fat. Further, it is thought that HPH-15 has a preventive or therapeutic effect against obesity caused by excessive accumulation of fat, for example, the subcutaneous fat obesity.

### [Example 11] Blood Test Value in Mouse

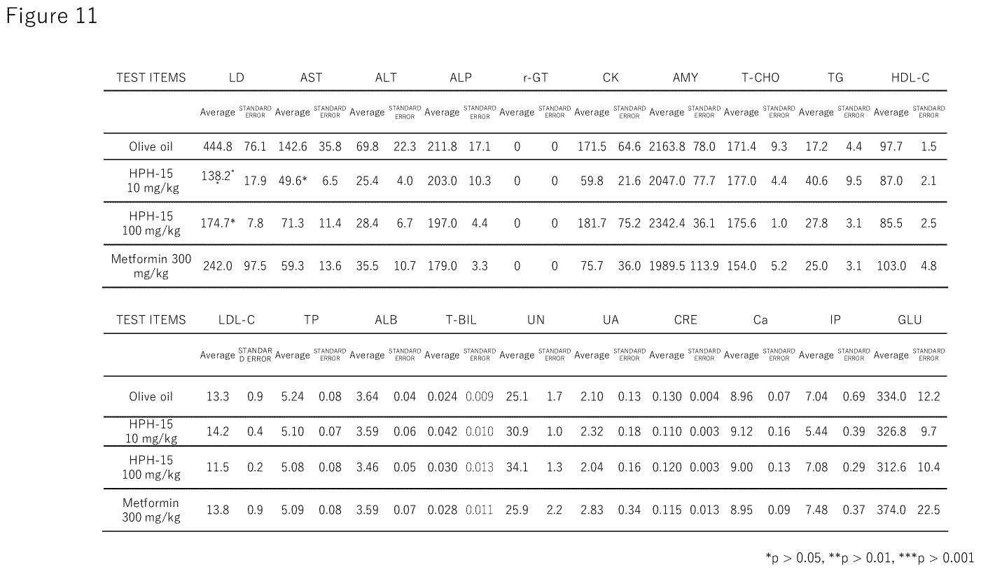

High-fat diet-induced obesity model mice were prepared in the same manner as in Example 7. Then, 200 mL of olive oil (Kenei Pharmaceutical Co., Ltd.) as a solvent, HPH-15 (10 mg/kg or 100 mg/kg, 200 mL) dissolved in olive oil, or metformin (300 mg/kg, 200 mL) was orally administered once a day for 28 days. After administration of each for 28 days, the blood from the high-fat diet-induced obesity model mice was examined at the Kumamoto Mouse Clinic (Kumamoto National University Corp.). The results are shown in FIG. 11.

As shown in FIG. 11, when HPH-15 was administered, an LD value and an AST value were significantly lower than those of metformin, and an ALT value was also lower than that of metformin, confirming that HPH-15 had a greater effect on improving liver function than metformin. On the other hand, no differences were observed in other test items compared to when olive oil was administered. No toxicity was observed by using HPH-15.

### [Example 12] H&E Staining of Mouse Tissue

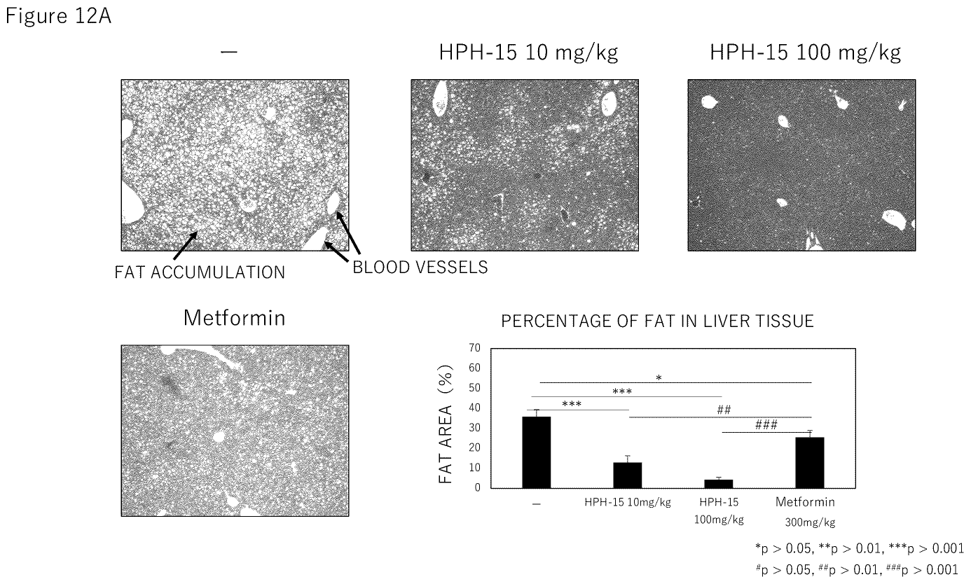

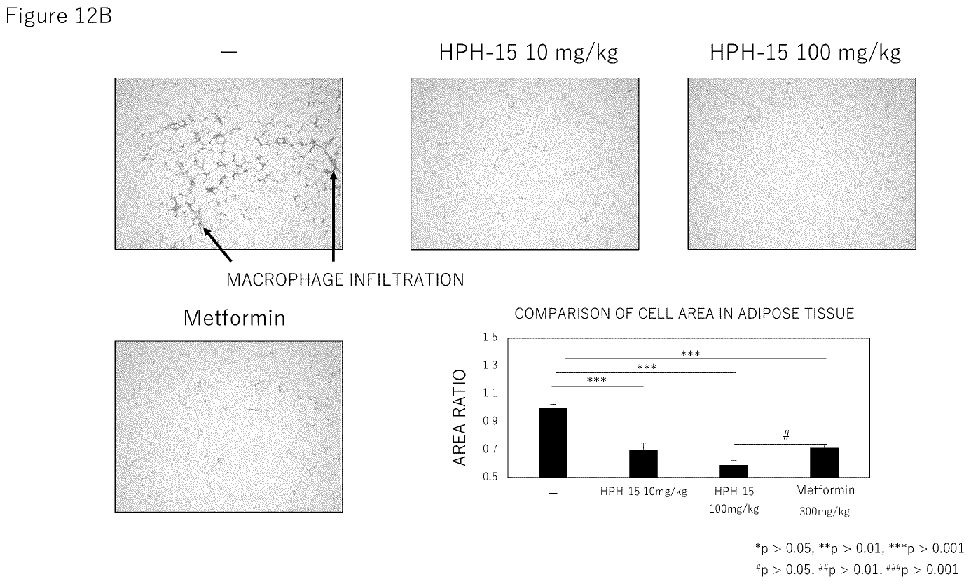

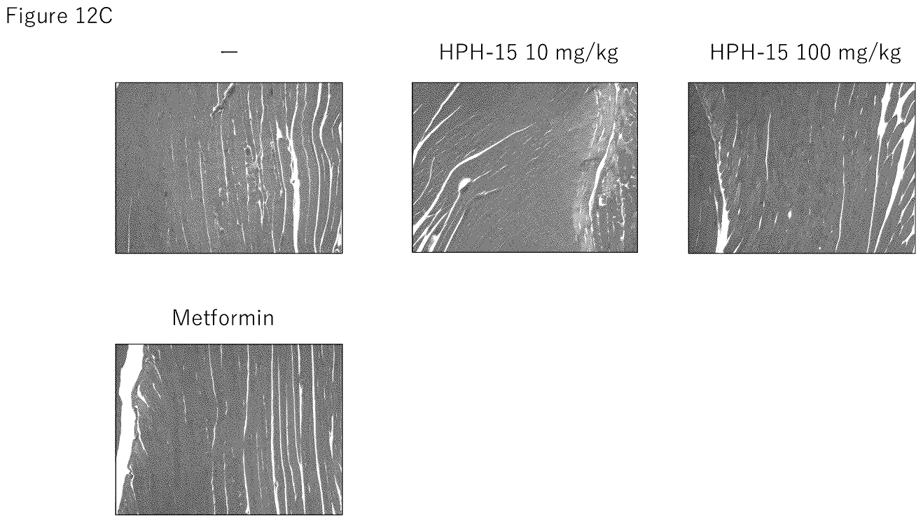

High-fat diet-induced obesity model mice were prepared in the same manner as in Example 7. Note that the high-fat diet-induced obesity model mice develop liver damage and are thus used as a liver disease model. Then, HPH-15 (10 mg/kg or 100 mg/kg, 200 mL) dissolved in olive oil or metformin (300 mg/kg, 200 mL) was orally administered once a day for 28 days. Olive oil alone was administered as a control. After administration of each for 28 days, the liver, epididymis, and muscle of the high-fat diet-induced obesity model mice were stained with H&E and observed using a microscope. The results of H&E staining of the liver are shown in FIG. 12A, the results of H&E staining of the epididymis are shown in FIG. 12B, and the results of H&E staining of the muscle are shown in FIG. 12C.

As shown in FIG. 12A and FIG. 12B, HPH-15 showed an effect of significantly reducing the percentage of fat in the liver tissue compared to the control and metformin administration. Furthermore, HPH-15 showed an effect of reducing the cell area ratio in the epididymal adipose tissue, that is, an effect of reducing the size of adipocytes. In particular, when 100 mg/kg of HPH-15 was administered, almost no fat was visible, confirming that the liver tissue and the epididymal adipose tissue were in a clean state. Note that administration of metformin only slightly reduced the fat area compared to control. Based on this result and the results shown in FIG. 5A and FIG. 5B above, it is thought that HPH-15 is effective in preventing or treating fatty liver and obesity. Further, in diabetic patients, nonalcoholic fatty liver disease (NAFLD), such as nonalcoholic steatohepatitis (NASH) or nonalcoholic fatty liver (NAFL), is caused by insulin resistance. In the diabetic patients, NASH or NAFL tends to become severe, and the fatty liver tends to progress to cirrhosis. Based on the above results, HPH-15 has the effect of reducing the percentage of fat cells and the fat area ratio, and is therefore considered to be effective in the prevention or treatment of NAFLD, such as NASH or NAFL. Note that the infiltration of macrophages observed in the control in FIG. 12B is due to the inflammatory reaction caused by the thickening of the adipose tissue. It is known that macrophage infiltration leads to liver fibrosis. Since HPH-15 does not cause macrophage infiltration, HPH-15 may also have an effect of reducing liver fibrosis.

Further, as shown in FIG. 12C, no tissue changes or toxicity was observed in the muscle.

### [Example 13] Activation of p-AMPK in Mouse Tissue

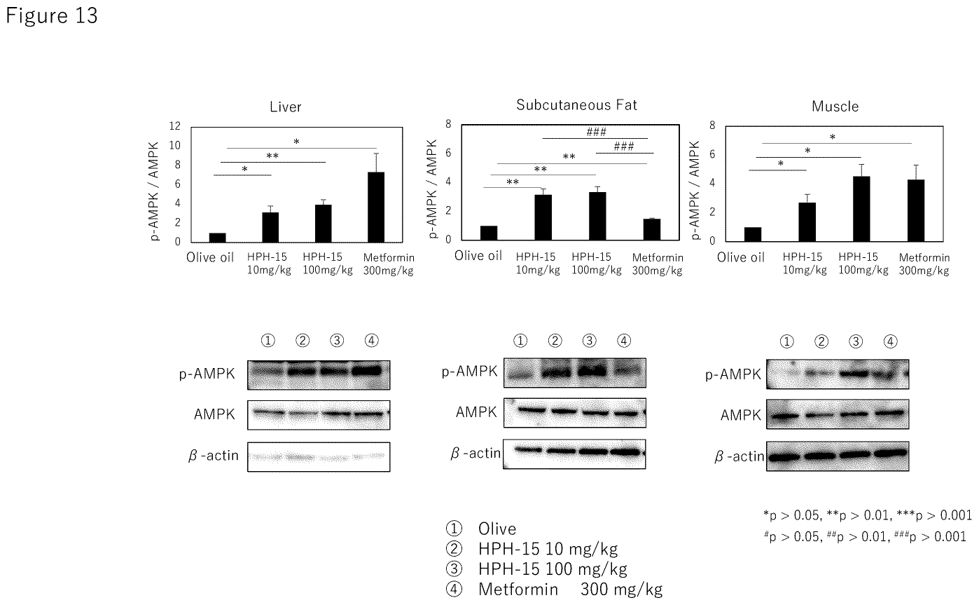

High-fat diet-induced obesity model mice were prepared in the same manner as in Example 7. Then, HPH-15 (10 mg/kg or 100 mg/kg, 200 mL) dissolved in olive oil or metformin (300 mg/kg, 200 mL) was orally administered once a day for 28 days. Olive oil alone was administered as a control. After 28 days of administration of each, equal amounts (50 mg) of tissue pieces were collected from the liver, subcutaneous fat, and muscle tissues of the high-fat diet-induced obesity model mice, transferred to 1.5 mL Eppendorf tubes, and washed twice with PBS. After washing, the tissue pieces were transferred to BIOMASHER II, 500 mL of RIPA buffer was added, and the tissue pieces were homogenized on ice. After homogenization, the tissue pieces were centrifuged (10,000 rpm, 4° C., 5 min) and the supernatant was obtained as a protein solution. The concentration of the proteins thus obtained was corrected based on the protein concentration measured by the BCA method using bicinchoninic acid (BCA), thereby preparing samples for Western blotting. Western blotting was performed in the same manner as in Example 1 described above, and the amount of p-AMPK/AMPK was calculated. The results are shown in FIG. 13.

As shown in FIG. 13, p-AMPK/AMPK was increased in the liver, subcutaneous fat, and muscle in the high-fat diet-induced obesity model mice. This confirmed that the use of HPH-15 activated AMPK in the high-fat diet-induced obesity model mice. In particular, it was confirmed that HPH-15 activated AMPK twice or more as compared to metformin in the subcutaneous fat.

### [Examples 14] Sirius Red Staining and Collagen Amount in Mouse Tissue

High-fat diet-induced obesity model mice were prepared in the same manner as in Example 7. Then, HPH-15 (10 mg/kg or 100 mg/kg, 200 mL) dissolved in olive oil or metformin (300 mg/kg, 200 mL) was orally administered once a day for 28 days. Olive oil alone was administered as a control. After 28 days of administration of each, the liver and the epididymal fat of the high-fat diet-induced obesity model mice were stained with Sirius Red and observed using a microscope. The results of staining of the liver (Liver) and the epididymal adipose tissue (Epididymal fat) are shown in FIG. 1 4A.

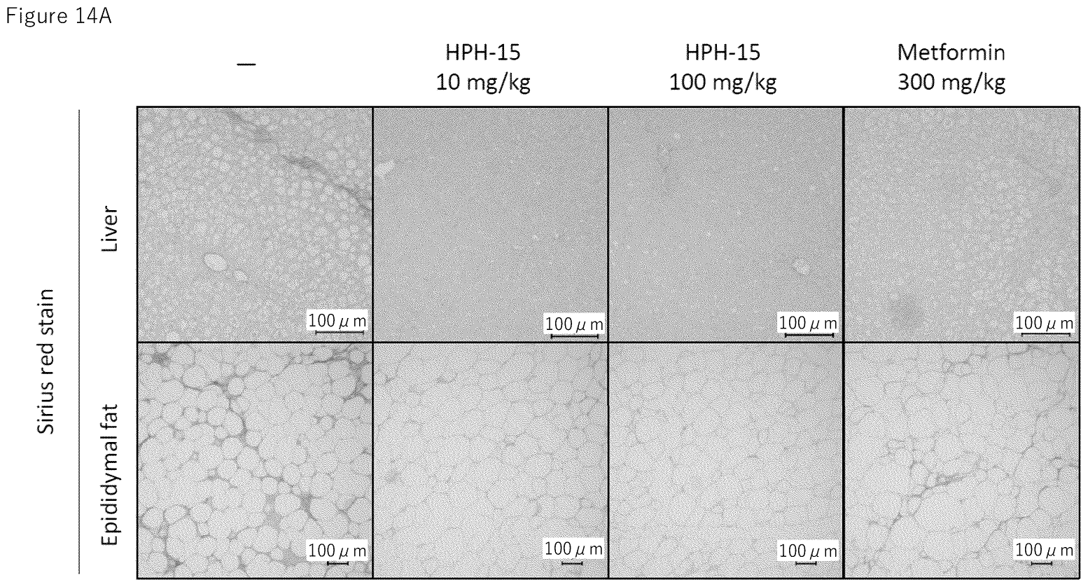

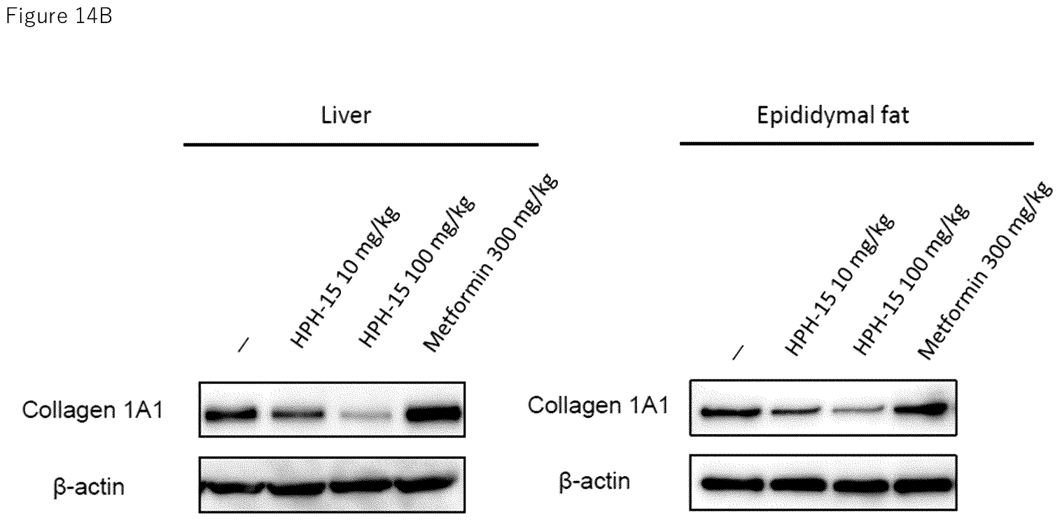

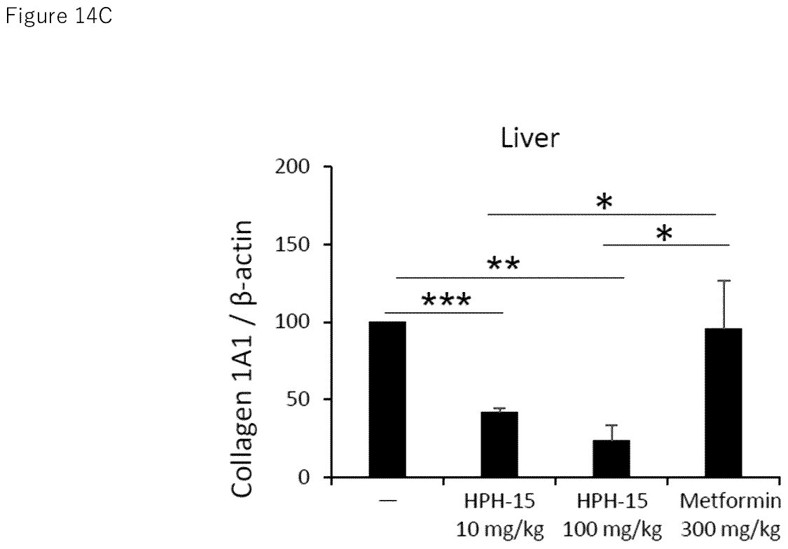

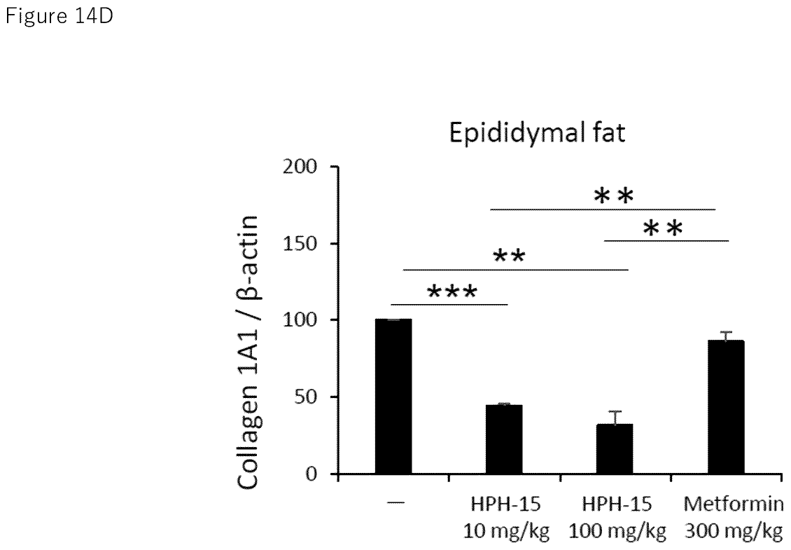

Further, high-fat diet-induced obesity model mice were prepared in the same manner as in Example 7. Then, HPH-15 (10 mg/kg or 100 mg/kg, 200 mL) dissolved in olive oil or metformin (300 mg/kg, 200 mL) was orally administered once a day for 28 days. Olive oil alone was administered as a control. After 28 days of administration of each, equal amounts (50 mg) of tissue pieces were collected from the liver and epididymal adipose tissues of the high-fat diet-induced obesity model mice, transferred to 1.5 mL Eppendorf tubes, and washed twice with PBS. After washing, the tissue pieces were transferred to BIOMASHER II, 500 mL of RIPA buffer was added, and the tissue pieces were homogenized on ice. After homogenization, the tissue pieces were centrifuged (10,000 rpm, 4° C., 5 min) and the supernatant was obtained as a protein solution. The concentration of the proteins thus obtained was corrected based on the protein concentration measured by the BCA method using bicinchoninic acid (BCA), thereby preparing samples for Western blotting. Western blotting was quantified using ImageJ to calculate the amount of collagen. The results are shown in FIGS. 14B to 14D.

As shown in FIGS. 14A to 14D, it was confirmed that the amount of collagen in the liver and epididymal adipose tissues was strongly reduced by the administration of HPH-15 at 10 mg/kg or 100 mg/kg. On the other hand, no collagen-reducing effect was confirmed with metformin.

1-5. (canceled)

6. A method of preventing or treating a disease selected from the group consisting of type 2 diabetes, a complication of type 2 diabetes, and a fat-related disease, comprising:
administering to a subject in need thereof an effective amount of a pharmaceutical composition comprising a compound represented by formula (1) or a salt thereof as an active ingredient:

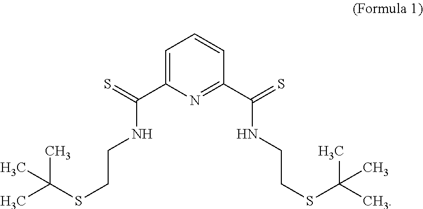 [SMILES: CC(C)(C)SCCNC(=S)c1cccc(C(=S)NCCSC(C)(C)C)n1]

7. The method according to claim 6, wherein the disease is type 2 diabetes or the complication of type 2 diabetes, and the complication of type 2 diabetes is diabetic retinopathy, diabetic macular edema, neovascular glaucoma, diabetic nephropathy, diabetic foot ulcer, or diabetic neuropathy caused by type 2 diabetes.

8. The method according to claim 6, wherein the disease is type 2 diabetes or the complication of type 2 diabetes, and the complication of type 2 diabetes is diabetic retinopathy, diabetic macular edema, neovascular glaucoma, diabetic foot ulcer, or diabetic neuropathy caused by type 2 diabetes.

9. The method according to claim 6, wherein the disease is type 2 diabetes.

10. The method according to claim 6, wherein the disease is the fat-related disease, and the fat-related disease is fatty liver disease, subcutaneous fat obesity, visceral fat obesity, hyperlipidemia, atherosclerosis, abdominal aortic aneurysm, coronary artery disease, macroangiopathy, cerebral infarction, or ischemic heart disease.

11. The method according to claim 10, wherein the fatty liver disease is nonalcoholic steatohepatitis (NASH) or nonalcoholic fatty liver (NAFL).
